SEQUENCE LISTING (4 sequences)

SEQ ID NO: 1 - protein, 525 aa, Homo sapiens [sequence omitted]

SEQ ID NO: 2 - protein, 342 aa, Homo sapiens
MPLFTANPFEQDVEKATNEYNTTEDWSLIMDICDKVGSTPNGAKDCLKAIMKRVNHKVPHVALQALTLLGACVANCGKIFHLEVCSRDFATEVRAVIKNKAHPKVCEKLKSLMVEWSEEFQKDPQFSLISATIKSMKEEGITFPPAGSQTVSAAAKNGTSSNKNKEDEDIAKAIELSLQEQKQQHTETKSLYPSSEIQLNNKVARKVRALYDFEAVEDNELTFKHGEIIIVLDDSDANWWKGENHRGIGLFPSNFVTTNLNIETEAAAVDKLNVIDDDVEEIKKSEPEPVYIDEDKMDRALQVLQSIDPTDSKPDSQDLLDLEDICQQMGPMIDEKLEEIDR

SEQ ID NO: 3 - DNA, 1833 bp, Homo sapiens
  CDS (111)..(1685)
tgaaggccgaagcggagcggtctagggagccgcggccgcgggtcacccggcgggtagcagttgctgagtgtcagctagacagcagcgactagggctcgggcgccggcgagatgcctttgttcaccgccaaccccttcgagcaagacgtggaaaaagccacgaatgagtacaacactacagaagattggagtcttattatggacatatgtgacaaagttggaagtactcctaatggagcgaaagattgcctaaaagccataatgaaaagggtaaatcataaggttccacatgttgctctgcaagcactaactcttcttggggcttgtgtggcaaactgtggaaagatatttcatttagaagtatgttcccgtgattttgcaacagaagtacgtgctgtgattaaaaataaggcacatcctaaagtatgtgaaaaactgaaatctttaatggtggagtggtcagaagaatttcagaaggaccctcagtttagtctgatatctgcaactattaaatctatgaaagaagaaggaattacttttcctccagcaggttctcagactgtctcagctgctgccaagaatggtacgtcatcgaacaaaaacaaagaggatgaagacatagctaaagctattgaattatcgctgcaagaacagaaacagcaacacacagaaacaaaatccttatatccatcttcagaaattcagttaaataataaggttgcacggaaagtgagagctttatatgattttgaagctgttgaggacaatgaactcacctttaaacatggtgaaataattattgttttggatgacagtgatgccaattggtggaaaggagaaaatcacagaggaataggacttttcccatccaattttgtaacaactaatttaaacatagagactgaggcagcggctgtggacaaattgaatgtaattgatgatgatgtggaggaaattaagaaatcagagcctgagcctgtttatatagatgaggataagatggatagagccctgcaggtacttcagagtatagatccaacagattcaaaaccagactcccaagaccttttggatttagaagatatctgccaacagatgggtccaatgatagatgaaaaacttgaagaaattgataggaagcattcagaattgtctgaattgaatgttaaagtcctggaagctctggaactatataacaaattggtgaatgaagcaccagtgtactcagtctattcaaagctccaccctccagcacattacccacctgcatcatctggggttccaatgcagacatatccagttcaatcacatggtggaaactatatgggtcagagcattcaccaagtaactgttgcccaaagctatagcctaggacccgatcaaattggtccactgagatctctgcctccaaatgtgaattcctcagtgacagcacagcctgctcaaacttcatatttaagcactggacaagacactgtttccaatcctacttatatgaaccagaactctaacctacagtcagctactggtacaactgcttacacacagcaaatggggatgtctgtggatatgtcatcttatcagaacactacttccaatttgcctcaactggcaggctttccggtgacagttccagctcatccagttgcacagcagcacacaaattaccatcagcagcctctcctttagaaacaaatcaagcattttcttgaaagccttcataagtgtattattcagtccttgtgataccaacctgaaaatattaaaacgaatttccagcacaactggcaaaaggaccatgaataaataaagcacaaaaacctctcttattcta

SEQ ID NO: 4 - DNA, 1368 bp, Homo sapiens
  CDS (111)..(1136)
tgaaggccgaagcggagcggtctagggagccgcggccgcgggtcacccggcgggtagcagttgctgagtgtcagctagacagcagcgactagggctcgggcgccggcgagatgcctttgttcaccgccaaccccttcgagcaagacgtggaaaaagccacgaatgagtacaacactacagaagattggagtcttattatggacatatgtgacaaagttggaagtactcctaatggagcgaaagattgcctaaaagccataatgaaaagggtaaatcataaggttccacatgttgctctgcaagcactaactcttcttggggcttgtgtggcaaactgtggaaagatatttcatttagaagtatgttcccgtgattttgcaacagaagtacgtgctgtgattaaaaataaggcacatcctaaagtatgtgaaaaactgaaatctttaatggtggagtggtcagaagaatttcagaaggaccctcagtttagtctgatatctgcaactattaaatctatgaaagaagaaggaattacttttcctccagcaggttctcagactgtctcagctgctgccaagaatggtacgtcatcgaacaaaaacaaagaggatgaagacatagctaaagctattgaattatcgctgcaagaacagaaacagcaacacacagaaacaaaatccttatatccatcttcagaaattcagttaaataataaggttgcacggaaagtgagagctttatatgattttgaagctgttgaggacaatgaactcacctttaaacatggtgaaataattattgttttggatgacagtgatgccaattggtggaaaggagaaaatcacagaggaataggacttttcccatccaattttgtaacaactaatttaaacatagagactgaggcagcggctgtggacaaattgaatgtaattgatgatgatgtggaggaaattaagaaatcagagcctgagcctgtttatatagatgaggataagatggatagagccctgcaggtacttcagagtatagatccaacagattcaaaaccagactcccaagaccttttggatttagaagatatctgccaacagatgggtccaatgatagatgaaaaacttgaagaaattgataggtgagatttttcgtgcaaggaataaaaagtatagttttggggggttcacttgtgttttggcaaacagtaaagtagggaaagtttttgtggaactgtacaattcatggaaattgtttttcagtttcccaggcttttaacaaatttatagcaaggtggtgctttttatcttgtttttgtttttctaaaaaacaagctctgacaagttttattaaataaaaattttgcatggctta

(19) United States
(12) Patent Application Publication
(10) Pub. No.: US 2002/0115203 A1
(43) Pub. Date: Aug. 22, 2002

(54) Human proteins STAM2a and STAM2b, and cDNAs encoding these molecules

(76) Inventors: Kazuo Sugamura, Miyagi (JP); Kazuhiro Endo, Miyagi (JP)

Correspondence Address: WENDEROTH, LIND & PONACK, L.L.P., 2033 K STREET N. W., SUITE 800, WASHINGTON, DC, 20006-1021, US

(21) Appl. No.: 09/942,845

(22) Filed: Aug. 31, 2001

Related U.S. Application Data
(63) Continuation-in-part of application No. 09/807,843.
Application No. 09/807,843 is a 371 of international application No. PCT/JP99/05756, filed on Oct. 19, 1999.

(30) Foreign Application Priority Data
Oct. 19, 1998 (JP) 1998-296465

(51) Int. Cl. C12N 15/00; C07H 21/04; C07K 16/28; C07K 14/715

(52) U.S. Cl. 435/320.1; 530/350; 536/23.5; 530/388.22

(57) ABSTRACT

This application provides human proteins STAM2a and STAM2b respectively containing the amino amid sequences of SEQ ID No. 1 and 2 which are novel signal transducers interacting with a cytokine signal transducer AMSH. This application also provides human gene encoding these proteins, their cDNAs respectively containing the nucleotide sequences of SEQ ID Nos. 3 and 4, and antibodies against STAM2a and STAM2b.

### TECHNICAL FIELD

The present invention relates to human proteins STAM2a and STAM2b, and the cDNAs encoding these molecules. More particularly, the present invention relates to novel signal transduction adapter molecules, STAM2a and STAM2b, human genes encoding these proteins, their cDNAs, and the antibodies against these proteins.

### BACKGROUND ART

A variety of cells having different functions should collaborate with each other for expression of higher biological functions such as hematopoietic, immunological and nervous systems. Communication among the cells is essential for their collaborations. Cytokines are known to be the proteins responsible for intercellular communication, and include interleukin (IL)-1 to 18, colony stimulation factors (CSFs), interferons (IFNS) and several chemokines.

Signals are generated by binding of the cytokines to specific receptors on the cell membrane, and survival, proliferation, differentiation and functional expression of the cells are controlled by signal transduction. Accordingly, dysfunction of cytokine-receptor signal transduction pathways result in collapse of the immunological and hematopoietic systems to cause severe infectious diseases, cancers and autoimmune diseases.

For the reasons described above, it is quite important to elucidate the intracellular signal transduction pathways mediated by the cytokine/cytokine receptor system. This is important, in order to appreciate the basic phenomena such as proliferation and differentiation of the cells, pathogenesis, diagnosis and therapeutic intervention of various diseases at the molecular levels.

The inventors of the present invention have isolated the genes of “common γ-chain” commonly included in a plurality of the cytokine receptors, and have made a great contribution in elucidating the structure and function of the cytokine receptors. Of particular elucidation is that the γ-chain is an essential subunit for functional expression of IL-2, IL-4, IL-7 and IL-9, and abnormality in the γ-chain causes impairment of early development of T-cells via dysfunction of IL-7 (Science, 262:1874-1877, 1993; Int. Immunol., 6:1451-1454, 1994; Science, 263:1453-1454, 1994; Eur. J. Immunol., 25:3001-3005, 1995).

Recently, the inventors of the present invention have identified “STAMs” as novel signal molecules concerning signal transduction in the proliferating cells by the cytokines, and found that these STAMs are present in the downstream of the IL-2/GM-CSF receptor and directly associate with JAK3/2. The inventors also found that STAMs play an important role in expression of c-myc and signal transduction for DNA synthesis (Immunity, 6:449-457, 1997).

While several important mechanisms of the intracellular signal transduction pathway by binding of the cytokines to the receptors have been made clear, additional novel molecules should be identified for elucidating the overall structure and function of the intracellular signal transduction pathway, because plural molecules are thought to be continuously and synergetically involved in the signal transduction pathway and to manifest final functional expression by constructing a so-called cascade.

### DISCLOSURE OF INVENTION

Accordingly, the object of the present invention is to provide adapter molecules similar to the STAMs that have been found by the inventors of the present invention.

Another object of the present invention is to provide a gene for these novel molecules and their cDNAs, and antibodies against these novel molecules.

The present invention for solving the above problems provides human proteins STAM2a and STAM2b having the amino acid sequences of SEQ ID Nos. 1 and 2, respectively.

The present invention also provides human genes encoding the human proteins STAM2a STAM2b described above; cDNAs of the human genes which have the nucleotide sequences of SEQ ID Nos. 3 and 4, respectively; and DNA fragments comprising partial sequences in the nucleotide sequences of SEQ ID Nos. 3 and 4, respectively.

The present invention further provides recombinant vectors containing the cDNAs or the DNA fragments described above, and antibodies against the human proteins STAM2a and STAM2b.

### BEST MODE FOR CARRYING OUT THE INVENTION

The procedures for obtaining the human proteins STAM2a and STAM2b of the present invention, and cDNAs for these proteins, as well as the procedures for identifying the functions of these proteins will be described first.

The human proteins STAM2a and STAM2b of the present invention are novel proteins obtained by the steps of isolating a sequence highly homologous to 5′-teminus of human STAM cDNA from the existing ESTs (Expression Sequence Tag), preparing an oligonucleotide probe based on the sequence, isolating respective cDNA clones by screening a human PHA stimulating leukocyte cDNA library, expressing these cDNA clones in a host cell, and isolating and purifying the novel proteins.

The human protein STAM2a has the sequence of SEQ ID No. 1 which consists of 525 amino acids and is encoded in the 111th to 1685th of SEQ ID No. 3 showing the nucleotide sequence of cDNA thereof. The protein has a molecular weight of 68 kDa and contains SH3 and ITAM regions as in STAM. STAM2a is 50.1% homologous to STAM. The human protein STAM2b has, on the other hand, the sequence of SEQ ID No. 2 consisting of 342 amino acids and is encoded in the 111th to 1136th of cDNA of SEQ ID No. 4. The protein STAMb2 may be conjectured to be a splicing variant of STAM2a in which the c-terminal including the ITEM region of STAM2a is deleted.

These proteins STAM2a and STAM2b are thought to have similar functions to STAM as adapter molecules in the signal transduction pathway involved in STAM. This suggestion may be confirmed by the following observations:

(1) STAM2a associates with Jak2 and Jak3 via the ITAM region;

(2) STAM2b does not associate with Jak2 and Jak3;

(3) A SH3 domain deleted variant of STAM2a suppresses DNA synthesis stimulated by IL-2 and GM-CSF; and

(4) Expression of c-myc gene stimulated by IL-2 and GM-CSF is accelerated by forced expression of STAM2a, however STAM2b does not have such acceleration activity.

The human proteins STAM2a and STAM2b can be obtained by using current method for isolating from human organs and cell lines. Preparing the peptides by chemical synthesis based on the amino acid sequences provided by the present invention, or a method for producing the proteins by the recombinant DNA technology using the cDNA fragments provided by the present invention. When the protein STAM2a or STAM2b is obtained by the recombinant DNA technology, for example, RNA is prepared by in-vitro transcription from the vector containing the cDNA of SEQ ID No. 3 or 4, and the protein may be expressed through in-vitro translation using the RNA as a template. Otherwise, by recombining an expression vector with the translation region of the cDNA by a known method in the art, the proteins encoded by cDNA may be obtained in a large scale in E. coli, bacillus subtilis, yeast, animal cells and plant cells.

When the proteins according to the present invention are produced by expression of DNA by in-vitro translation, the cDNA or its translation region is recombined into a vector containing a RNA polymerase promoter, and the recombinant cDNA is added in an in-vitro translation system such as a rabbit reticulocyte dyalizate or wheat germ extract containing a RNA polymerase for the promoter. Examples of the RNA polymerase promoter include T7, T3 and SP6. The vectors containing these polymerase promoter are, for example, pKA1, pCDM8, pT3/T7 18, pT7/3 19 and pBluescript II.

For large scale production of the protein encoded by cDNA in microorganisms such as E. coli, an expression vector is constructed by inserting cDNA according to the present invention or its translation region into an expression vector having an origin suitable for microorganism, a promoter, ribosome binding cite, cDNA cloning cite and terminator, followed by cultivation of the transformant obtained after transformation of the host cell with the expression vector. Protein molecules containing arbitrary regions may be obtained by permitting the cDNA to express by adding an initiating codon and a stop codon before and after an arbitrary translation region. Otherwise, only the desired protein portion may be obtained by allowing the protein to express as a fused protein with other proteins, followed by leaving the fused protein with an appropriate protease. Examples of the expression vector in E. coli include pUC, pBluescript II, pET and pGEX expression system vectors.

For producing the protein accord to the present invention in eukaryotic cells such animal cells, the cDNA or its translation region is inserted into an expression vector for the eukaryotic cells containing a promoter, splicing regions and poly(A) additional site to introduce the recombinant vector into the eukaryotic cells. Examples of the expression vectors include pKA1, pCDM8, pSVK3, pMSG, pSVL, pBK-CMV, pBK-RSV, EBV vector, pRS and pYES2. While mammal culture cells such as monkey kidney cells COS7 and Chinese hamster ovary cells CHO, budding yeast, dividing yeast silkworm cells and African clawed frog egg cells are usually used as the eukaryotic cells, any eukaryotic cells may be used so long as the cells can express the proteins according to the present invention. The methods known in the art such as an electroporation method, a calcium phosphate method, a liposome method and a DEAE dextran method may be used for introducing the expression vector into the eukaryotic cells.

Separation procedures known in the art may be combined for isolating and purifying the desired protein from the cultivation product after expressing the protein according to the present invention in microorganisms or eukaryotic cells. The separation and purification methods include, for example, treatment with urea or a denaturation agent, ultrasonic treatment, enzymatic digestion, salting out and solvent precipitation, dialysis, centrifugation, ultrafiltration, gel filtration, SDS-PAGE, isoelectric electrophoresis, ion-exchange chromatography, hydrophobic chromatography, affinity chromatography and reversed phase chromatography.

Peptide fragments (more than five amino acid residues) containing any partial amino acid sequences of the amino acid sequence represented by the sequence numbers 1 or 2 are included in the proteins STAM2a and STAM2b according to the present invention. These peptide fragments may be also used for antigens for preparing antibodies. Fused proteins with other arbitrary proteins are also included in the proteins according to the present invention.

The gene according to the present invention is the human gene encoding the foregoing proteins, and can be isolated from the existing genome libraries using cDNA or its partial sequence as a probe.

cDNA according to the present invention can be obtained by screening the cDNA library derived from the human cells by colony or plaque hybridization method known in the art using the oligonucleotide synthesized based on the nucleotide sequence of cDNA of SEQ ID No. 3 or 4. Alternatively, cDNA according to the present invention may be also prepared from mRNA isolated from human cells by the RT-PCR method using a synthetic oligonucleotide that can hybridize to both terminus of the cDNA fragment as a primer.

Polymorphism due to individual difference is often observed in the human gene. Accordingly, cDNAs containing addition or deletion of one or plural nucleotides, and/or substitution with other nucleotides in SEQ ID No. 3 or 4 are also included in cDNA according to the present invention.

Likewise, proteins containing addition or deletion of one or plural amino acids, and/or substitution with other amino acids caused by alteration of cDNA may be included in the proteins according to the present invention, provided that the protein comprises protein activity of the protein having the amino acid sequence of SEQ ID No. 1 or 2.

The DNA fragment according to the present invention may include any cDNA fragments (10 bp or more) containing any partial nucleotide sequences of SEQ ID No. 3 or 4. DNA fragments comprising sense strand and antisense strand may be categorized into the DNA fragments according to the present invention. These DNA fragments can be used for a probe for gene diagnosis.

Antibodies against the proteins according to the present invention may be obtained as polyclonal or monoclonal antibodies by a conventional method using the protein itself or a partial peptide thereof as an antigen.

### EXAMPLE

Applied on the PD-10 column was 5 mg of KLH (Keyhole Limpets Hemocyanin) after treating with a chemical cross-link agent (3-meleimidobenzoic acid N-hydroxy-succinimide) to obtain MBS linked KLH. A synthetic peptide of STAM2a (the amino acid number 17-29 represented by the sequence number 1) to the C-terminal of which cysteine was added was prepared, and a KLH linked STAM2a peptide was obtained by allowing the peptides to react with MBS linked KLH. A rabbit was immunized with this peptide as an antigen to obtain an antiserum. After precipitation of the antiserum with 40% saturation ammonium sulfate, the antiserum was purified by a protein A affinity column to obtain an IgG antibody.

### INDUSTRIAL APPLICABILITY

The present invention as hitherto described in detail provides novel adapter molecules related to the cytokine based signal transduction pathway and gene engineering materials. These molecules and gene engineering materials are useful for developing diagnostic and therapeutic methods of human diseases due to dysfunction of cytokine based signal transduction pathwaysuch as severe infectious diseases, cancers and autoimmune diseases.

1. A human protein STAM2a having the amino acid sequence of SEQ ID No. 1.

2. A human protein STAM2b having the amino acid sequence of SEQ ID No. 2.

3. A human gene encoding the human protein STAM2a of claim 1.

4. cDNA of the human gene of claim 3, which is STAM2a cDNA having the nucleotide sequence of SEQ ID No. 3.

5. cDNA of the human gene of claim 3, which is STAM2b cDNA having the nucleotide sequence of SEQ ID No. 4.

6. A DNA fragment comprising a partial sequence in the nucleotide sequence of SEQ ID No. 3.

7. A DNA fragment comprising a partial sequence in the nucleotide sequence of SEQ ID No. 4.

8. A recombinant vector containing the STAM2a cDNA of claim 4 or the DNA fragment of claim 6.

9. A recombinant vector containing the STAM2b cDNA of claim 5 or the DNA fragment of claim 7.

10. An antibody against the human protein STAM2a of claim 1.

11. An antibody against the human protein STAM2b of claim 2.
